(19) United States
(12) Patent Application Publication
(10) Pub. No.: US 2023/0348414 A1
(43) Pub. Date: Nov. 2, 2023

(54) THE PROCESS OF PREPARATION OF ANTHRANILAMIDES

(71) Applicant: Adama Makhteshim Ltd., Beer Sheva (IL)

(72) Inventors: Gal SUEZ, Dimona (IL); Hagit FORCKOSH, Beer Sheva (IL); Yanai GOLUB, Omer (IL); Jie LIE, Nanjing (CN); Bob CHEN, Shanghai (CN); Regev PARNES, Kibbutz Mishmar Hanegev (IL)

(73) Assignee: Adama Makhteshim Ltd., Beer Sheva (IL)

(21) Appl. No.: 18/009,072

(22) PCT Filed: Jun. 8, 2021
(86) PCT No.: PCT/CN2021/098904

§ 371 Date: Dec. 8, 2022

Related U.S. Application Data
(60) Provisional application No. 63/035,865, filed on Jun. 8, 2020.

(51) Int. Cl. C07D 401/04

(52) U.S. Cl. CPC C07D 401/04

(57) ABSTRACT

Provided is a process for preparing arthropocidal athramnilamides of formula (Ia) and (Ib) by reaction of acyl chloride and the corresponding anthranilic acid amides wherein the process is carried out in the absence of acid scavenger and hydrochloric acid formed is distilled out from the reaction mixture during the process.

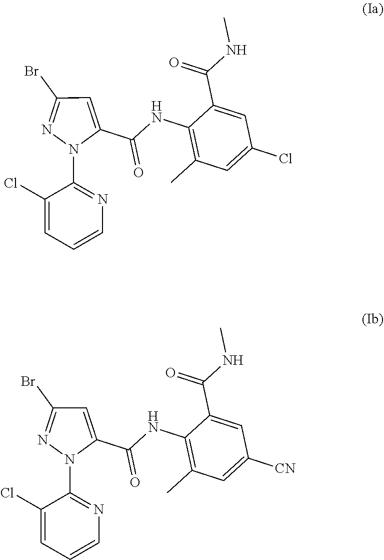 [SMILES: CNC(=O)c1cc(Cl)cc(C)c1NC(=O)c1cc(Br)nn1-c1ncccc1Cl ; CNC(=O)c1cc(C#N)cc(C)c1NC(=O)c1cc(Br)nn1-c1ncccc1Cl]

### FIELD OF THE INVENTION

This invention relates to the process of preparation of arthropocidal anthranilamides by reaction of acyl chloride and the corresponding anthranilic acid amides wherein the process is carried out in the absence of acid scavenger.

### BACKGROUND OF THE INVENTION

Arthropodicidal anthranilamides have wide range of applications and are previously disclosed as active pesticides in PCT patent applications such as WO 2001/070671, WO 2003/015519, WO 2003/015518 and WO 2004/067528. General methods of their preparation are disclosed for example in PCT Patent Publications WO 2001/070671, WO 2006/062978, WO 2003/016283, WO 2004/011447 and WO 2003/015519.

It has already been disclosed that anthranilamides of formula I(a) and I(b) could be obtained by reacting the corresponding anthranilic acids or amides of anthranilic acids with heteroaryl carboxylic acids or their acyl halides in the presence of acid scavengers, usually, amines as disclosed in WO 2006/062978, WO 2019/207595 and WO 2021/033172. However, these preparative methods still must be improved for safe economic commercial operation. In particular, the acid scavengers used in the previously recited processes belong to organic amines class and should be present in molar excess toward the starting pyrazole carboxylic acid. The resulting hydrochloride salts of said acid scavengers are hardly removed from the process and their presence in most cases leads to the formation of “cake” which cannot be easily removed from the reaction vessel. In addition, the presence of organic base as an acid scavenger in the aforementioned processes, affects the cyano group in the anthranilamides containing cyano-substituents and reduces the yield and purity of final anthranilamide. It would be highly desirable to have an improved process for the production of the anthranilamides of formula (Ia) and/or (I(b) which is suitable for industrial use, highly efficient, low-cost, environmentally friendly, and provides a high yield in a short reaction time, thereby overcoming the deficiencies of the prior art. The present subject matter provides such a process.

### SUMMARY OF THE INVENTION

The present invention provides a process of preparation of anthranilamides of formula (Ia) or (Ib)

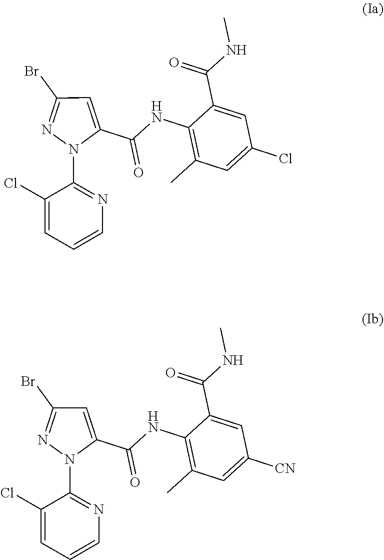 [SMILES: CNC(=O)c1cc(Cl)cc(C)c1NC(=O)c1cc(Br)nn1-c1ncccc1Cl ; CNC(=O)c1cc(C#N)cc(C)c1NC(=O)c1cc(Br)nn1-c1ncccc1Cl]

comprising reaction of compound of formula (II)

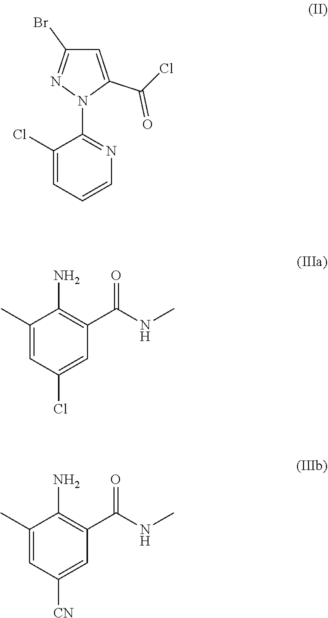

with the compound of formula (IIIa) or (IIIb) in the presence of aromatic solvent wherein the process is carried out in the absence of acid scavenger.

The present invention further provides a process of preparation of anthranilamides of formula (Ia) or (Ib) wherein the hydrochloric acid formed is distilled out from the reaction mixture during the process.

### DETAILED DESCRIPTION OF THE INVENTION

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one skilled in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.”

Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

Certain compounds of this invention can exist as one or more stereoisomers. The various stereoisomers include enantiomers, diastereomers, and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

Accordingly, the compounds of the invention may be present as a mixture of stereoisomers, individual stereoisomers, or as an optically active form.

The present invention provides technology useful for the successful and convenient preparation of anthranilic acid amides in the absence of acid scavenger. Moreover, said technology includes distillation out of the hydrochloric acid formed during the preparation of anthranilic acid amides

According to an embodiment, the present invention concerns the process of preparation of anthranilamides of formula (Ia)

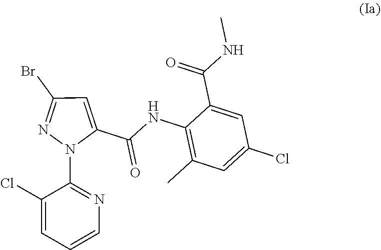 [SMILES: CNC(=O)c1cc(Cl)cc(C)c1NC(=O)c1cc(Br)nn1-c1ncccc1Cl]

comprising reaction of compound of formula (II)

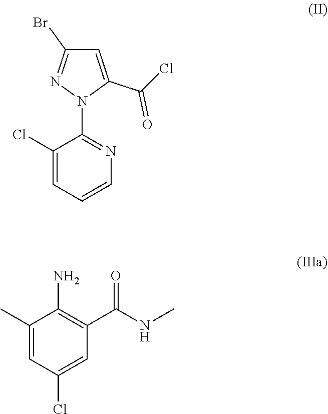

with the compound of formula (IIIa) in the presence of aromatic solvent, wherein the process is carried out in the absence of acid scavenger.

In purpose of the invention the acid scavenger is referred to a chemical substance added to a reaction mixture in order to remove or de-activate acids generated in the process, for example, organic amines can serve as acid scavengers.

According to another embodiment, the present invention concerns the process of preparation of anthranilamides of formula (Ib)

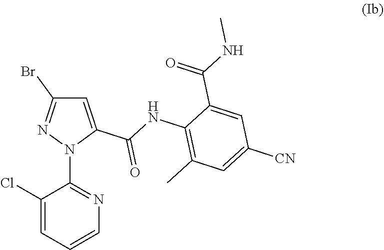 [SMILES: CNC(=O)c1cc(C#N)cc(C)c1NC(=O)c1cc(Br)nn1-c1ncccc1Cl]

comprising reaction of compound of formula (II)

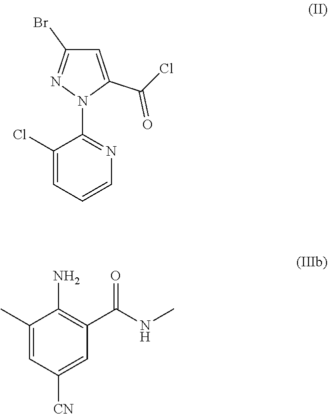

with the compound of formula (IIIb) in the presence of aromatic solvent, wherein the process is carried out in the absence of acid scavenger.

Pyrazolecarboxylic acid chloride of formula (II) can be prepared from the corresponding pyrazole carboxylic acids using known methods, for example by reaction of said carboxylic acids with chlorination agents like thionyl chloride, oxalyl chloride and the like as recited in WO 2001/070671, WO 2003/015519, WO 2003/015518, WO 2003/016283.

The corresponding starting pyrazole carboxylic acid can be prepared using methods described in literature, for example in WO 2003/015519, WO 2003/016283, WO 2006/102025, WO 2013/076092.

Anthranilic acid amides (IIIa) and (IIIb) can be prepared according to the methods described for example in WO2003/016300, WO 2008/010897, WO 2013/007603.

In some embodiment, the process carried out wherein the molar ratio of compound of formula (II) to the compound of formula (IIIa) is about 1.2:1 to about 1:1.2.

In some another embodiment, the process carried out wherein the molar ratio of compound of formula (II) to the compound of formula (IIb) is about 1.2:1 to about 1:1.

In some embodiment, the process carried out “in the absence of acid scavenger” refers to the process including not more than 0.01 mole of acid scavenger on each 1 mole of pyrazole carboxylic acid of formula (II).

In some embodiment, the process carried out “in the absence of acid scavenger” refers to the process including not more than 0.05 mole of acid scavenger on each 1 mole of pyrazole carboxylic acid of formula (II).

In some embodiment, the process carried out “in the absence of acid scavenger” refers to the process including not more than 0.1 mole of acid scavenger on each 1 mole of pyrazole carboxylic acid of formula (II).

In another embodiment, the process carried out “in the absence of acid scavenger” refers to the process including not more than 0.5 mole of acid scavenger on each 1 mole of pyrazole carboxylic acid of formula (II).

For the purpose of the present invention, the acid scavenger refers to the chemical compound added to the reaction mixture which is able to remove or de-activate impurities and unwanted reaction products, in particular, hydrochloric acid formed during the process of preparation of anthranilamides of formula (Ia) and (Ib).

In some embodiments, the acid scavengers are referred to organic bases such as amines; e.g triethylamine, diethylamine, substituted or unsubstituted pyridine, picoline, lutidine and the like.

The process is suitably carried out in the aromatic solvent such as toluene, xylene, ethylbenzene, chlorobenzene and the mixtures thereof. Optionally, additional inert solvent could be used in the inventive process.

According to another embodiment the hydrochloric acid formed in the process is distilled out from the reaction mixture during the process.

In a class of this embodiment is the process wherein a distillation of hydrochloric acid is performed at a temperature in the range of from 75° C. to 100° C.

In a class of this embodiment is the process wherein a distillation of hydrochloric acid is performed at a temperature in the range of from 75° C. to 85° C. In a class of this embodiment is the process wherein a distillation of hydrochloric acid is performed at a temperature in the range of from 85° C. to 100° C.

In a class of this embodiment is the process wherein a distillation of hydrochloric acid is performed at a temperature of 80° C.

In a class of this embodiment is the process wherein a distillation of hydrochloric acid is performed at a temperature of 85° C.

In yet another embodiment, the distillation of hydrochloric acid is performed at a temperature of 90° C. to 100° C.

In a class of this embodiment is the process wherein a distillation of hydrochloric acid is performed at a pressure of 380 to 420 mm Hg.

In another embodiment the process of distillation of hydrochloric acid is performed at a pressure of 420 to 450 mm Hg.

In a class of this embodiment is the process wherein a distillation of hydrochloric acid is performed at a pressure of 380 to 400 mm Hg.

Without further elaboration, it is believed that one skilled in the art using the preceding description is suitable to utilize the present invention to its fullest extent. The following Examples are, therefore, to be construed as merely illustrative, and not limiting of the disclosure in any way whatsoever.

### EXAMPLES

### Example 1: Preparation of Cyanthraniliprole (Ib)

a) Preparation of 3-bromo-1-(3-chloropyridin-2-yl)-1H-pyrazole-5-carbonyl chloride (compound (II)): 3-bromo-1-(3-chloropyridin-2-yl)-1H-pyrazole-5-carboxylic acid (40 kg, 132 moles) and toluene (97 kg) were charged into a reactor and the resulting suspension was stirred and heated to 80° C. Thionyl chloride (20 kg, 168 moles) was added dropwise during 120 minutes to the above suspension. Then the resulting mixture was stirred for 1 hr. After reaction completion by HPLC, solvent was distilled off at 80° C. under reduced pressure of 450 mbar and 66 kg of toluene was added. After that, the final crude mixture was filtered and washed with 5-10 kg of toluene to obtain 3-bromo-1-(3-chloropyridin-2-yl)-1H-pyrazole-5-carbonyl chloride as 27% w/w toluene solution.
b) In another reactor, 2-amino-5-cyano-N,3-dimethylbenzamide (20 g, 102 mmol) was charged in 150 gr toluene and azeotropic distillation was done to remove water. Then, the resulting solution was heated to 75° C. under vacuum of 420 mbar and the solution of 3-bromo-1-(3-chloropyridin-2-yl)-1H-pyrazole-5-carbonyl chloride prepared in a) was added dropwise to the 2-amino-5-cyano-N,3-dimethylbenzamide in toluene under vacuum. Then the reaction mass was stirred for 2 h. During the stirring, fresh toluene is added (380 gr) under vacuum. After reaction completion by HPLC, the precipitated solid is filtered. The resulting solid was dried overnight under vacuum at 60° C. to afford 46.4 g cyantraniliprole of formula (Ia) as light brown solid (yield—94.8%), purity by HPLC—98.8%.

### Example 2: Comparative Example for the Preparation of Cyantraniliprole (Ib) in the Presence of 3-Picoline as Acid Scavenger

2-amino-5-cyano-N,3-dimethylbenzamide (20 g, 1.02 mmol) and toluene (193 gr) were mixed and heated to 80° C. under vacuum of 400 mbar. Then the solution of 3-bromo-1-(3-chloropyridin-2-yl)-1H-pyrazole-5-carbonyl chloride in toluene (115.6 gr, 98 mmol) prepared in Example 1 a) was added dropwise to the reaction mass during 120 minutes while vacuum distillation occurred. After that vacuum was removed, 3-picoline was added (2.4 gr, 26 mmol) and the reaction mixture was stirred for 6 h, then the reaction mixture is cooled to ambient temperature and the solid is filtered and washed with toluene and dried under vacuum at 70° C. overnight to afford cyantraniliprole of formula (Ib) as 28.2 g (51% yield) as light brown solid with purity by HPLC 84.4%.

### Example 3: Preparation of Chlorantraniliprole (Ia)

2-amino-5-chloro-N,3-dimethylbenzamide (22 g, 0.111 mol mol) was charged in 220 gr toluene and the mixture has been heated up to 85° C. under pressure of 450 mbar. The solution of 3-bromo-1-(3-chloropyridin-2-yl)-1H-pyrazole-5-carbonyl chloride in toluene (0.107 mol, 27% w/v) prepared in Example 1 a) has been added dropwise was added dropwise to the reaction mass during 120 minutes, while vacuum distillation occurred. Then 50 gr of toluene was added dropwise and distillation continued for additional one hour. After that 3 gr (0.038 mol) of pyridine added dropwise to the reaction mixture and the reaction cooled to r.t. The crude product was filtered, washed with toluene and then with methanol and dried to afford chlorantraniliprole of formula (Ia) as 46.5 gr (90% yield) as off-white solid with purity by HPLC 98.0%.

1. A process of preparation of anthranilamides of formula (Ia) or (Ib) 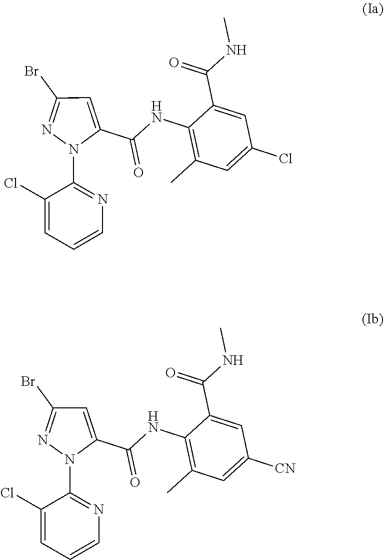 [SMILES: CNC(=O)c1cc(Cl)cc(C)c1NC(=O)c1cc(Br)nn1-c1ncccc1Cl ; CNC(=O)c1cc(C#N)cc(C)c1NC(=O)c1cc(Br)nn1-c1ncccc1Cl]
comprising reaction of compound of formula (II) 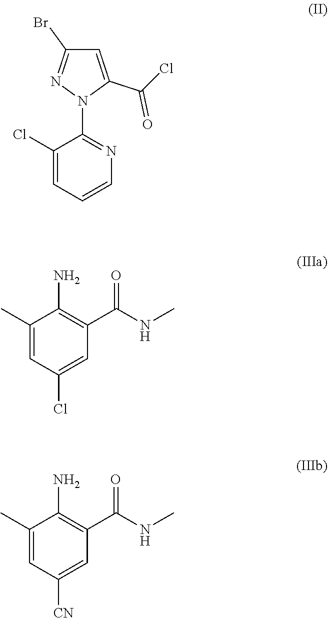
with the compound of formula (IIIa) or (IIIb) in the presence of aromatic solvent wherein the process is carried out in the absence of acid scavenger.

2. The process according to claim 1 wherein the aromatic solvent is selected from the group consisting of toluene, xylene, ethylbenzene, chlorobenzene and the mixtures thereof.

3. The process according to claim 1 wherein the hydrochloric acid formed is distilled out from the reaction mixture during the process.

4. The process according to claim 1 wherein distillation of hydrochloric acid is performed at a temperature in the range of from 75° to 100° C. and at a pressure of 380 to 450 mm Hg.

5. The process according to claim 1 wherein distillation of hydrochloric acid is performed at a temperature in the range of from 75° to 85° C.

6. The process according to claim 1 wherein distillation of hydrochloric acid is performed at a pressure of 420 to 450 mm Hg.
